(19) United States
(12) Patent Application Publication
(10) Pub. No.: US 2024/0216285 A1
(43) Pub. Date: Jul. 4, 2024

(54) INTRACELLULAR TARGETED NANOCARRIERS: TOWARDS CONTROLLED DRUG DELIVERY FOR NONINVASIVE NEUROREGENERATION TREATMENT

(71) Applicant: The Texas A&M University System, College Station, TX (US)

(72) Inventor: Ya Wang, Los Altos, CA (US)

(73) Assignee: The Texas A&M University System, College Station, TX (US)

(21) Appl. No.: 18/288,289

(22) PCT Filed: Apr. 26, 2022
(86) PCT No.: PCT/US2022/026398

§ 371 Date: Oct. 25, 2023

Related U.S. Application Data
(60) Provisional application No. 63/179,942, filed on Apr. 26, 2021.

(51) Int. Cl. A61K 9/50; A61K 9/51; A61K 31/203; A61K 41/00

(52) U.S. Cl. CPC A61K 9/5094; A61K 9/5146; A61K 31/203; A61K 41/0042

(57) ABSTRACT

In an embodiment, the present disclosure pertains to a nanocarrier having a shell and a core disposed within the shell. In some embodiments, the shell includes a functionalized surface. In an additional embodiment, the present disclosure pertains to a method of drug delivery. In general, the method includes administering a nanocarrier to a subject, targeting, by the nanocarrier, an area in the subject, and releasing a composition having the drug to the area. In some embodiments, the nanocarrier has a shell and a core disposed within the shell. In some embodiments, the shell includes a functionalized surface.

### CROSS-REFERENCE TO RELATED APPLICATIONS

This application claims priority to, and incorporates by reference the entire disclosure of, U.S. Provisional Patent Application No. 63/179,942 filed on Apr. 26, 2021.

### STATEMENT REGARDING FEDERALLY SPONSORED RESEARCH

This invention was made with government support under 1751435 awarded by the National Science Foundation. The government has certain rights in the invention.

### TECHNICAL FIELD

The present disclosure relates generally to nanocarriers and more particularly, but not by way of limitation, to intracellular targeted nanocarriers directed towards controlled drug delivery for noninvasive neuroregeneration treatment.

### BACKGROUND

This section provides background information to facilitate a better understanding of the various aspects of the disclosure. It should be understood that the statements in this section of this document are to be read in this light, and not as admissions of prior art.

Effectively delivering potential therapeutic agents to the central nervous system remains a challenge. In particular, many of the potential drugs for neurodegenerative diseases, including, for example, genes, growth factors, and neuroprotective drugs, have a very short half-life. It is therefore difficult to sustain their therapeutic effect due to the rapid metabolization process. Additionally, most of the drugs have poor water-solubility and limited blood brain barrier permeability which restricts them from reaching the targeted area with sufficient concentrations. Smart intracellular targeted nanocarriers are highly desirable for controlled drug delivery for non-invasive neuroregeneration. However, current nanocarriers either lack sufficient control of loading or delivering of therapeutic agents, or are too complicated to be assembled efficiently.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a nanocarrier having a shell and a core disposed within the shell. In some embodiments, the shell includes a functionalized surface.

In an additional embodiment, the present disclosure pertains to a method of drug delivery. In general, the method includes administering a nanocarrier to a subject, targeting, by the nanocarrier, an area in the subject, and releasing a composition having the drug to the area. In some embodiments, the nanocarrier has a shell and a core disposed within the shell. In some embodiments, the shell includes a functionalized surface.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates a schematic representation of a nanocarrier according to an aspect of the present disclosure.

FIG. 2A illustrates analysis of particles size distribution of superparamagnetic iron oxide (SPIO) nanoparticles (NPs) (11.7±1.7 nm) based on transmission electron microscopy (TEM) imaging.

FIG. 2B illustrates analysis of particles size distribution of superparamagnetic iron oxide-gold (SPIO-Au) NPs (19.6±3.7 nm) based on TEM imaging.

FIG. 3A illustrates normalized ultraviolet-visible (UV-Vis) spectra of the stand-alone SPIO-NPs, SPIO-Au NPs functionalized with rhodamine B-(polyethylene glycol)-SH (RhB-PEG-SH), and the stand-alone RhB-PEG-SH. The peak absorbance value as 1.

FIG. 3B illustrates the fluorescence spectrum of the SPIO-Au NPs functionalized with pyrene (Py)-PEG-SH.

FIG. 4 illustrates correlograms of SPIO-Au NPs, SPIO-Au-RhB, SPIO-Au-RhB-porous coordination cage (PCC)-2, SPIO-Au-Py, and SPIO-Au-Py-PCC-3 in deionized water at different time points.

FIG. 5A illustrates the zeta potential measurement of SPIO-Au NPs with acid neutralization for acetic acid amount from 20 μL to 500 μL at the concentration of 0.175 mol/L for every 127 μg of SPIO-Au NPs.

FIG. 5B illustrates the zeta potential measurement of SPIO-Au NPs with acid neutralization and further functionalization with Py and PCC-3 showing that the zeta potential is changed from negative to positive. Sample 1 is original SPIO-Au; Sample 2 is acid washed SPIO-Au; Sample 3 is acid washed SPIO-Au-Py; and Sample 4 is acid washed SPIO-Au-Py-PCC-3.

FIG. 6 illustrates the relative absorbed amount of Au in neuron cells treated with SPIO-Au NPs and SPIO-Au NPs functionalized with Py, RhB, Py-PCC-3, and RhB-PCC-2. Cells without NPs were used as the control group with nearly 0 Au absorption. *p<0.05; **p<0.01; ***p<0.001. Original data set is listed in Table 4.

FIG. 7 illustrates the cellular viability determined from flow cytometry for PC-12 neuron cells treated with SPIO-Au NPs and SPIO-Au NPs functionalized with Py, RhB, Py-PCC-3, and RhB-PCC-2. The cells without NPs were used as control group. *p<0.05; **p<0.01; ***p<0.001.

FIG. 8A illustrates an ultraviolet (UV) light absorbance spectra of standard retinoic acid (RA) solution and the top solution after the centrifugation process to separate the PCC-3 functionalized SPIO-Au NPs loaded with RA as precipitate.

FIG. 8B illustrates an accumulated release profile of RA from SPIO-Au-Py-PCC-3.

FIG. 9A illustrates an experimental setup of the temperature measurement of SPIO-Au NPs solutions according to aspects of the present disclosure.

FIG. 9B illustrates a temperature profile of SPIO-Au NPs in deionized (DI) water with different concentrations. Volume is set to 150 μL.

FIG. 9C illustrates a temperature profile of SPIO-Au NPs with different volumes. Concentration is set to 200 μg/mL.

FIG. 10A illustrates an experimental procedure for in vitro drug release: (1) treated by laser for 10 min period; (2) incubated until the next time point; (3) collected released RA for analysis; and (4) refilling the tube with fresh DI water for the next treatment.

FIG. 10B illustrates a cumulative release profile of RA in DI water from SPIO-Au-Py-PCC NPs (200 μL, 200 μg/mL).

FIG. 10C illustrates an initial release profile of RA in DI water from SPIO-Au-Py-PCC NPs (500 μL, 200 μg/mL).

FIG. 11 illustrates cell viability measured from flow cytometry for PC-12 cells treated with different concentrations of SPIO-Au-Py-PCC NPs. Cells without treatment by NPs were used as the control group.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

As smart intracellular targeted nanocarriers are highly desirable for controlled drug delivery for non-invasive neuroregeneration, in various aspects, the present disclosure pertains to smart nanocarriers composed of superparamagnetic iron oxide-gold (SPIO-Au) core-shell nanoparticles (NPs), porous coordination cages (PCCs), and thiol (SH) containing molecules (e.g., pyrene-(rhodamine B)-(polyethylene glycol)-SH; Py(RhB)-PEG-SH) as bridges. More specifically, the SPIO-Au cores, modified by two types of PCCs: PCC-2 with negative and PCC-3 with positive net charges, respectively, are suitable for intracellular-targeting and various drug encapsulation by tuning the surface charge of SPIO-Au core through neutralizing the citrate anions before the surface modification. Transmission electron microscopy (TEM) confirmed the cellular internalization of PCCs functionalized SPIO-Au NPs and the stand-alone SPIO-Au NPs via direct translocation across cellular membranes, without compromising the cell integrity. Particularly, the pyrene (Py) functionalization remarkably enhances the cellular uptake of SPIO-Au NPs compared with RhB functionalized counterparts and the stand-alone SPIO-Au NPs. The nanocarriers are not toxic to PC-12 neuron-like cells for concentrations up to 20 μg/ml. The nanocarriers exhibit effective drug loading capacity and a sustainable drug release pattern of retinoic acid (RA) for up to 48 hours in vitro. These nanocariers have great potential to deliver different types of drugs for non-invasive neuroregeneration by customizing their surface charge and functionalization.

As briefly discussed, effectively delivering potential therapeutic agents to the central nervous system (CNS) remains a challenge. In particular, many of the potential drugs for neurodegenerative diseases, including, for example, genes, growth factors, and neuroprotective drugs, have a very short half-life. It is therefore difficult to sustain their therapeutic effect due to the rapid metabolization process. Additionally, most of the drugs have poor water-solubility and limited blood brain barrier (BBB) permeability which restricts them from reaching the targeted area with sufficient concentrations. To address these issues, a promising way to extend the circulation time and achieve controllable drug release is to use the colloidal NPs as drug carriers. In particular, the surface of these nanocarriers can be modified to improve BBB penetrability via receptor-mediated pathways and extend the circulation time of therapeutic agents.

Among these therapeutic molecules, retinoic acid (RA) is known to play a role in promoting neuronal differentiation. RA can induce the expression of mRNA and the protein transcription factors, such as, for example, Nurr1 and Pitx3, which are responsible for dopaminergic neuronal survival and development. It is also reported that RA can enhance the expression of P2X₂ receptors in rat PC-cells, which plays a role in synaptic transmission between neurons. RA can also induce the differentiation of embryonic stem cells into neural cells and advance the neuronal differentiation of mouse neuroblastoma cells. It is believed that RA can initiate a biochemical program of neuronal differentiation in PC-12 cells, however, unlike nerve growth factors, a fully differentiated phenotype with neurite extension still has not been obtained. The administration of RA is also a significant challenge because of its short half-life, low water solubility, and rapid metabolization by cells. In particular, the release of RA has to be carefully controlled to avoid the undesired side-effects. The use of NPs as a nanocarrier can potentially overcome these obstacles and enable the controlled release of RA.

To date, several nanocarriers have been developed for the delivery of RA. For example, some type of polymeric NPs where employed to deliver RA intracellularly for inducing the differentiation of neural stem cells (NSC) with minimal side effect on cellular viability and proliferation. It was found that polyethylenimine/dextran sulfate NPs loaded NPs loaded RA worked much more efficiently than free RA at very low concentrations (10 μg/mL) on enhancing the NSC proliferation and protecting them from ischaemia death. Some have used metal NPs as an RA loading vehicle, such as encapsulating RA with small interfering RNAs and superparamagnetic iron oxides (SPIOs) by cationic polymers to control the differentiation of NSC. Others have used dendrimer coated SPIO NPs to load RA for pancreatic cancer. However, these nanocarriers either lack sufficient control of loading or delivering RA, or are too complicated to be assembled efficiently. Therefore, a facile assembled nanoplatform is needed for controllable loading and release of RA, and a generalized strategy of loading and release for many types of drugs by tailoring the surface functionalization is also needed. With a build-in component with high contrast for imaging, these types of platforms can also enable the live tracking of the drug release.

Regarding the nanocarriers of RA, PCCs composed of metal clusters and organic linkers are one of the potential options to encapsulate RA and realize the controllable RA release. These structures have been reported to have tailored electronic and chemical properties by changing the assembling elements, such as, for example, the type of metal clusters and organic linkers, and the surface functional groups. As an example, by replacing tert-butyl groups with anionic sulfate groups, the whole structure can be modified from a neutral to negative charge. Particularly, this porous structure is pH responsive, and therefore, can be used to carry guest small therapeutic molecules within these pores by strong electrostatic interactions and hydrophobic nature, and release them in a controllable way by adjusting the pH value. Presented herein are customized PCCs for versatile guest molecule encapsulation of rhodamine B, nile red, and camptothecin for anticancer treatment by adjusting the hydrophobic or electronic interactions between the host and the guest. It was found that the PCC drug carriers can penetrate the cytoplasmatic membrane by direction translocation, and show passive cell-organelle-targeting because of the dedicated surface affinity.

However, the in vitro and in vivo detections are barely possible because of the low contrast of some PCC structures for imaging, such as magnetic resonance imaging (MRI), X-ray, TEM, and computed tomography (CT) because of the low electron-density difference between PCCs and the tissue. Also, although PCCs have been reported as a potential drug carrier for anticancer treatment, the possibility of conjugating PCCs with RA for neuroregeneration has not been explored and there is a lack of sufficient information about the biocompatibility, toxicity, and cellular internalization of PCCs in neurons.

SPIO-Au NPs have been proved with excellent biocompatibility and uptake ability for neuron-like PC-12 cells in previous studies. SPIO-Au NPs also serve as an excellent dual-mode contrast agent in MRI and CT imaging by using gold nanoshells to reduce the saturation magnetization and improve X-ray absorption. The SPIO core of the NPs can be remotely moved towards targeting sites by adjusting the level of magnetic field to be applied. Therefore, SPIO-Au NPs have advantages as a potential smart nanocarrier considering their excellent magnetic property from the iron oxide core and the outstanding biocompatibility, biostabilities, and tunable surface function from the Au shell. Additionally, the benefit of Au NPs as a potential nanomedicine on the differentiation, growth, and maturation of neurons has been proven before in biomaterial scaffolds. The scaffolds with the decoration of Au NPs of 10 nm were shown to promote longer neurite outgrowth with lower number of branches, which can be potentially used for neuronal regeneration, such as repairing longer gaps in spinal cord injuries. Intraspinal delivery of 40 nm Au NPs functionalized with PEG were tested and found to be beneficial for recovery after spinal cord injury of mice, such as the recovery of the motor function and the enhanced survival of motor neuron with no adverse effects like the loss of body weight and ill health. Au NPs were also shown to enhance the differentiation of embryonic stem cells into dopaminergic neurons via the activation of mammalian target of rapamycin (mTOR)-mediated signaling pathway by examining the phosphorylation level of p70s6k through western blot analysis.

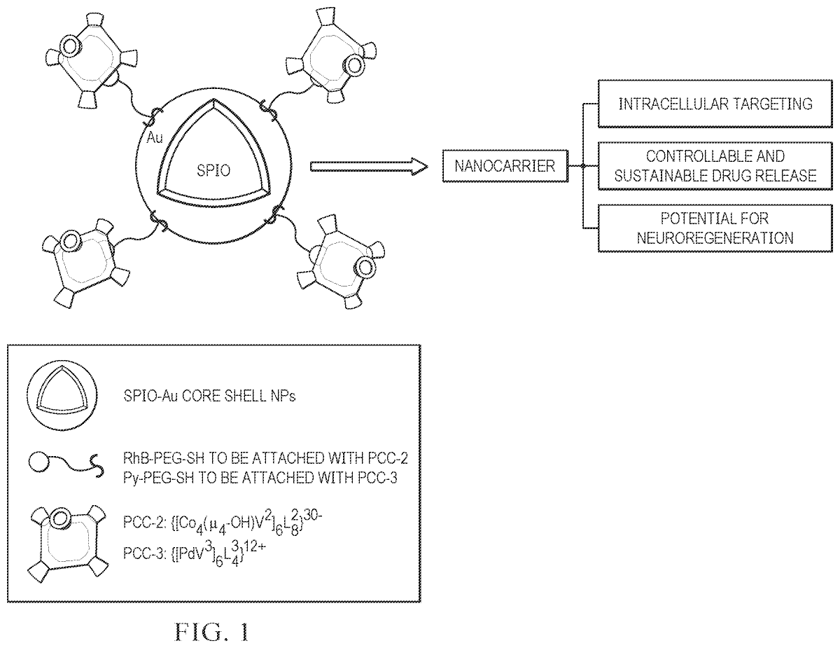

The aforementioned advantages of SPIO-Au NPs and PCCs as potential drug carriers provide motivation to explore a novel generalized drug delivery nanocarrier integrating SPIO-Au NPs and PCCs with thiol containing molecules (e.g., SH-PEG-Py (RhB)) as a bridge. As shown in FIG. 1, in this type of structure, the thiol group on one end will link the surface of Au NPs due to the strong Au—S linkage, while the Py and RhB groups on the other side attract PCC-3 and PCC-2, respectively, due to the strong electrostatic force. Without being bound by theory, it is hypothesized that this single nanocarrier would possess outstanding contrast in bioimaging, tunable surface charge, and functionalization for controllable drug loading and release, as well as the enhanced intracellular targeting for efficient drug delivery. In this nanocarrier, the surface charge can be easily tuned by adjusting the amount and type of functional groups as well as the experimental conditions like pH. This feature provides better design and control of the effective drug loading for a specific drug with certain chemical and electrical properties. Additionally, the different types of surface functionalization of SPIO-Au NPs can affect the cellular uptake efficiency.

The present disclosure shows that the surface charge of this proposed nanocarrier can be manipulated to promote effective drug loading of RA by trapping the RA in the cage via electrostatic interactions. Sustainable RA release was observed. It is demonstrated herein that this nanocarrier has neglectable effect on the cell viability of PC-12 cells. The present disclosure also proves the intracellular targeting of this nanoplatform in PC-12 cells. Furthermore, the effect of different surface functionalization on cellular uptake efficiency is revealed, which promotes sufficient drug delivery into the cell organelles.

### WORKING EXAMPLES

Reference will now be made to more specific embodiments of the present disclosure and data that provides support for such embodiments. However, it should be noted that the disclosure below is for illustrative purposes only and is not intended to limit the scope of the claimed subject matter in any way.

Preparation of SPIO-Au NPs and PCCs. SPIO-Au NPs were synthesized using the seed growth method. Briefly, the SPIO cores were synthesized by the coprecipitation method. And the Au shell was formed at the surface of SPIO core by using hydroxylamine to reduce Au³⁺. PCC-2 and PCC-3 were synthesized using the solvothermal method. Briefly, PCC-2 was formed by the reaction among vertex ligand, panel ligand and CoCl₂. PCC-3 was prepared by the reaction between the vertex metal complex and the panel ligand.

Functionalization of SPIO-Au NPs with RhB(Py)-PEG-SH and PCCs. SPIO-Au NPs were first functionalized with RhB-PEG-SH (FL045003-1K, Biochempeg Scientific Inc.) and Py-PEG-SH (PG2-PNTH-2K, Nanocs Inc.), respectively, by suspending 366 μg SPIOAu NPs in 1 mL of deionized (DI) water solution with 100 μg RhBPEG-SH/Py-PEG-SH at room temperature for 24 hours. Then the thiol functionalized SPIO-Au NPs were isolated by centrifugation and washed by water at least 3 times. RhBPEG-SH functionalized SPIO-Au NPs were then suspended in 1 mL of dimethylsulfoxide (DMSO) solution and mixed with 25 μg PCC-2 at room temperature for 24 hours. The resultant was then centrifuged and washed by DMSO at least 3 times. On the other hand, Py-PEG-SH functionalized SPIO-Au NPs were suspended in DI water and mixed with 10 μg PCC-3 at room temperature for 24 hours. The resultant was then centrifuged and washed by water at least 3 times. To adjust the surface charge of PCC-3 functionalized SPIO-Au NPs for the purpose of loading RA, 0.175 M of acetic acid at the volume of 20, 100, 200, and 500 μL were added to every 127 μg of SPIO-Au NPs for 5 minutes to neutralize the surface charge of the stand-alone SPIO-Au NPs. The NPs were then centrifuged and washed 3 times to remove the extra acetic acid. After that, the NPs were functionalized by Py and PCC-3 following the same steps mentioned above.

TEM Imaging of SPIO Core and SPIO-Au NPs. To observe the morphology of the synthesized SPIO-Au NPs, TEM imaging was performed on a FEI Tecnai G2 F20 S-Twin field-emission scanning transmission electron microscope at an operating voltage of 200 kV. Briefly, 100 μL droplets of each sample were dropped onto a 400-mesh copper grid (Electron Microscopy Sciences, Hatfield, PA) and then left to dry in the air.

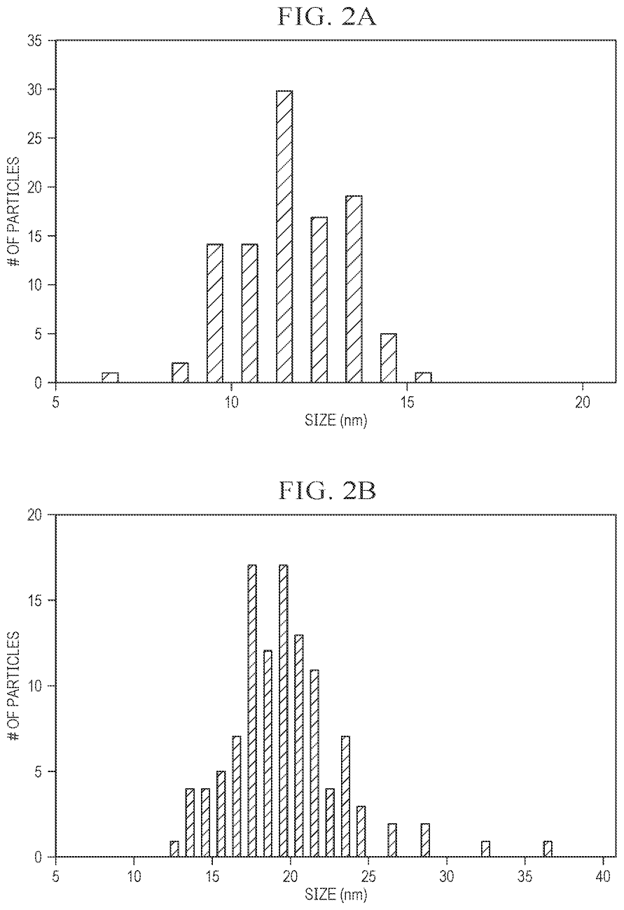

FIG. 2A shows analysis of particles size distribution of SPIO-NPs (11.7±1.7 nm) based on TEM imaging. FIG. 2B shows analysis of particles size distribution of SPIO-Au NPs (19.6±3.7 nm) based on TEM imaging.

The Functionalization of SPIO-Au NPs with RhB-PEG-SH—UV-VIS Light Absorbance Measurement. To verify the functionalization of SPIO-Au NPs with RhB-PEG-SH, the light absorption spectra of SPIO-Au NPs before and after the functionalization with thiol groups were recorded at wavelengths between 350 nm and 1000 nm at room temperature with a Shimadzu UV-2450 spectrophotometer (Shimadzu Corp.).

The Functionalization of SPIO-Au NPs with Py-PEG-SH—Fluorescence Measurement. To verify the functionalization of SPIO-Au NPs with Py-PEG-SH, the fluorescence signal of SPIO-Au NPs after the functionalization with Py-PEG-SH were recorded at nm excitation at room temperature with an RF-5301PC Spectrofluorophotometer (Shimadzu Corp.).

Surface Charge and Hydrodynamic Diameter Measurement of SPIO-Au NPs with Different Functionalizations. The zeta potential and the hydrodynamic diameter of SPIO-Au NPs before and after the functionalization with different thiol groups and PCCs was measured using a Zetasizer Nano ZS (Malvern Instruments Inc.). The zeta potential of SPIO-Au NPs before and after the neutralization with acetic acid at different amount, and the acid neutralized SPIO-Au NPs functionalized with Py and PCC-3 were tested as well using the same instrument.

The Quantification of Elemental Composition—Inductively Coupled Plasma Mass Spectrometry (ICP-MS). The SPIO-Au NPs before and after functionalization with PCCs were digested in mineral acid to bring Au, Fe, Co and Pd into solution. 2% nitric acid was used as an analytical matrix for ICP-MS. In detail, the NPs were centrifuged with the removal of top solution and digested by a mixture of HCl (0.3 mL, ˜30%) and HNO₃ (1.1 mL, ˜65%). The samples were then diluted into 2% nitric acid and tested on the NEXION® 300D inductively coupled plasma mass spectrometer (PerkinElmer, Inc.).

Cell Culture. PC-12 cells (from rat pheochromocytoma) obtained from American Type Culture Collection (ATCC) were cultured in ATCC modified Roswell Park Memorial Institute (RPMI)-1640 medium supplemented with 10% heat inactivated horse serum (HS), 5% fetal bovine serum (FBS) and 1% penicillin-streptomycin (medium and supplements were purchased from Gibco, Grand Island, NY). Cells were cultured in poly-L-lysine (PLL, P4707, Sigma-Aldrich, St. Louis, MO) coated Petri dishes in a humidified incubator under a 5% CO₂ atmosphere at 37° C. To induce cell differentiation, PC-12 cells were incubated in serum reduced media (1% heat inactivated HS and 0.5% FBS).

The Quantification of Gold Uptake—ICP-MS Analysis. PC-12 cells were seeded into CORNING® BIOCOAT™ 24-well plates pre-coated with collagen type IV and incubated for 1 day. Then the cells were washed with phosphate-buffered saline (PBS) and incubated with SPIO-Au NPs supplemented medium with different surface functionalizations (Groups: control (no NPs), SPIO-Au, SPIO-Au-Py, SPIO-Au-RhB, SPIO-Au-Py-PCC-3, and SPIO-Au-RhB-PCC-2). For all groups with NPs, the mass concentration of SPIO-Au is 20 μg/mL. After 1, 3, and 5 days, the cells were washed with PBS 3 times. Then the cells were digested by a mixture of HCl (0.3 mL, ˜30%) and HNO₃ (1.1 mL, ˜65%). Followed by the dilution into 2% nitric acid and tested on the NEXION® 300D inductively coupled plasma mass spectrometer (PerkinElmer, Inc.).

Cell Viability via Flow Cytometry. To examine the cytotoxicity of SPIO-Au NPs, PC-12 cells were seeded into CORNING® BIOCOAT™ 24-well plates precoated with collagen type IV and incubated for 1 day. Then the cells were washed with PBS and incubated with SPIO-Au NPs supplemented medium with different surface functionalizations (Groups: control (no NPs), SPIO-Au, SPIO-Au-Py, SPIO-Au-RhB, SPIO-Au-Py-PCC-3, and SPIO-Au-RhB-PCC-2). For all groups with NPs, the mass concentration of SPIO-Au is 20 μg/mL. After 1, 3, and 5 days, the media were removed and the cells were stained with SYTOX™ Green (1:30000 dilution from stock solution, Invitrogen) and SYTO™ 59 (1:1000 dilution from stock solution, Invitrogen) for 30 minutes with protection from light. Then the staining solution was removed, and the cells were washed 2-3 times using serum free media. The “green” labeled dead cells (by SYTOX™ Green) and the red labeled total cells (by SYTO™ 59) were then examined through an IX81 motorized inverted microscope (Olympus Corp.). Then the cells were trypsinized and re-suspended in serum free medium for fluorescence measurement through BD ACCURI™ C6 flow cytometer equipped with FL1 detector (553 nm) and FL3 detector (670 nm). All data was acquired at a flow rate of 14 μL/min with a minimum of 20,000 events detected.

TEM Image Analysis—Cellular Uptake. To study the cellular uptake of PCC functionalized SPIO-Au NPs, PC-12 cells were seeded onto 35 mm PERMANOX™ petri dishes at a density of 2×10⁵ cells per dish and incubated for 24 hours. Then the media was replaced by growth medium containing NPs (groups: SPIO-Au, SPIO-Au-Py-PCC-3, and SPIO-Au-RhB-PCC-2). For all groups with NPs, the mass concentration of SPIO-Au is 20 μg/mL. After 48 hours of incubation, cells were rinsed with serum-free media and 0.1M Cacodylate buffer. Cells were then fixed in 2% paraformaldehyde, 3% glutaraldehyde in 0.1M Cacodylate buffer (pH 7.4) at room temperature on the shaker for 1 hour and washed for 10 minutes with 0.1M Cacodylate buffer (pH 7.4) on shaker at room temperature 3 times. Followed by the dehydration in a graded series of ethyl alcohol and embedded in Epon 812 resin. Ultrathin sections (100 nm) were cut with an EM UC6 ultramicrotome (Leica Microsystems Inc.) and collected on formvar coated copper slot grids. Then these sections were analyzed by using a FEI transmission electron microscope, at an accelerating voltage of 80 kV.

Determination of the Loading Efficiency and the Release Profile of RA. 10 μL of RA in DMSO solution was added to the 1 mL of SPIO-Au-Py-PCC-3 solution with the concentration of 1274.7 μg/mL (SPIO-Au) for 48 hours. After then the solution was centrifuged, and the top solution was separated from the precipitate. The RA absorption spectra of the standard RA solutions (4.69-75 μg/mL; R²=0.9995) and the top solution was measured at 350 nm using an UV-2450 spectrophotometer (Shimadzu Corp.). The concentration of the free unbounded RA in the top solution was calculated using the standard curve according to the Beer-Lambert law that there is a linear relationship between the light absorbance and the concentration. The loading efficiency and the payload of NPs were calculated using the following equations:

$$Loading efficiency ( \% ) = ( Total feeded RA - free unbounded RA in the solution ) / ( Total feeded RA ) \times 100 \% \qquad Equation 1$$

$$Payload = ( Mass of RA loaded by NPs ) / ( mass of NPs ) \qquad Equation 2$$

To obtain the drug release profile at 3 hours, 6 hours, 12 hours, 24 hours, and 48 hours, the previous RA loaded SPIO-Au-Py-PCC-3 NPs (1274.7 μg of SPIO-Au) were suspended in 1 mL of DMSO in each tube. The samples were protected from light to prevent the photodegradation of RA. At successive time point, each tube of sample was centrifuged at 10000 rpm and the top solution were collected and measured by ultraviolet measurements at 350 nm. The RA content in the top solution were determined according to the standard curve of RA solutions (standard curve of RA solutions: 4.69-75 μg/mL; R²=0.9995).

Statistical Analysis. Results were analyzed based on at least three independent experiments and presented as mean t standard deviation. Statistical analysis was performed by using the analysis of variance (ANOVA) with Tukey post-hoc test to assess SPIO-Au treated groups with different surface functionalizations. A p-value less than 0.05 was considered as a significant difference.

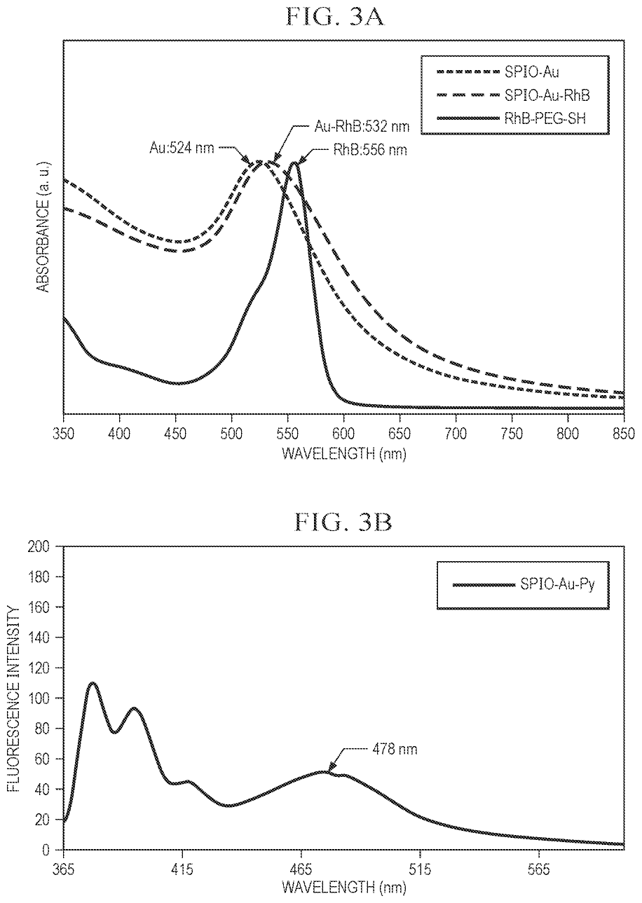

The Functionalization of SPIO-Au NPs with Thiol Groups. The TEM images showed the initial SPIO cores (D: 11.7±1.7 nm) before the Au coating, and the SPIO-Au NPs (D: 19.6±3.7 nm) synthesized by using the seed growth method. The morphology of the NPs after Au coating was uniform with a quasi-spherical shape and narrow size distribution. The light absorbance spectrum in FIG. 3 showed the typical peak of Au around 524 nm, which confirmed the formation of Au coating. The SPIO-Au NPs were then functionalized with RhB-PEG-SH and Py-PEG-SH, respectively, based on the strong Au—S linkage. By comparing the UV-Vis light absorbance spectrum of SPIO-Au NPs before and after the functionalization with RhB-PEG-SH, as shown in FIG. 3A, a peak shift was found from 524 nm to 532 nm. This change in the absorbance spectrum was mainly due to the surface attachment of RhB-PEG-SH on SPIO-Au NPs, since the RhB-PEG-SH exhibited a strong light absorbance at 556 nm. Unfortunately, a weak fluorescence signal was observed for RhB-PEG-SH functionalized NPs due to the fluorescence quenching effect. On the other hand, the fluorescence spectra in FIG. 3B confirmed the successful functionalization of SPIO-Au NPs with Py-PEG-SH by showing the typical fluorescence peak at 478 nm which came from the Py group. Zeta potential and hydrodynamic diameter results further confirmed the functionalization of SPIO-Au NPs with thiol groups. As shown in Table 1, the zeta potential was changed from −36.3 mV to −25.1 mV by the functionalization of SPIO-Au NPs by Py-PEG-SH with neutral charges, while the zeta potential was changed from −36.3 to −23.6 mV by the functionalization of SPIO-Au NPs by RhB-PEG-SH with positive charges. This change in zeta potential confirmed the replacement of the weakly attached negative citrate molecules by the neutral Py-PEG-SH groups and the positive RhB-PEG-SH groups respectively due to the strong Au—S linkage. The hydrodynamic diameter was also changed from 55.4 nm to 121.3 nm and 79.5 nm, for the functionalization of Py-PEG-SH and RhB-PEG-SH, respectively, which further confirmed the increase of the hydrodynamic size of NPs due to the functionalization with thiol groups with long chain. The much larger hydrodynamic diameter of Py-PEG-SH functionalization than RhB-PEG-SH functionalization was due to the larger molecular weight of Py-PEG-SH (2k Da of Py-PEG-SH vs. 1k Da of RhB-PEG-SH).

TABLE 1
The zeta potential measurement and hydrodynamic diameter for SPIO—Au NPs with different functionalization.

| | SPIO—Au | SPIO—Au—Py | SPIO—Au—Py—PCC-3 | SPIO—Au—RhB | SPIO—Au—RhB—PCC-2 |
|---|---|---|---|---|---|
| Zeta Potential | −36.3 | −25.1 | −14.4 | −23.6 | −29.8 |
| (mV) | | | | | |
| Hydrodynamic | 55.4 | 121.3 | 244.4 | 79.5 | 185.7 |
| Diameter (nm) | | | | | |

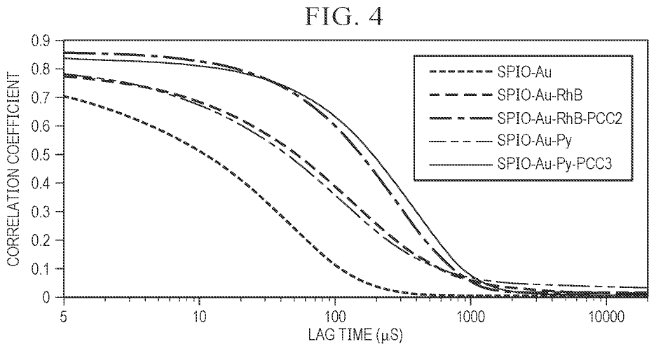

The Functionalization of Thiol Modified SPIO-Au NPs with PCC-2 and PCC-3. Given that thiol modified SPIO-Au NPs have negative surface charge, modifying them with negatively charged PCC-2 and positively charged PCC-3 will significantly change the overall surface net charge of them. Thus, the thiol functionalized SPIOAu NPs were further functionalized with PCC-2 and PCC-3 using RhB and Py as bridges, respectively. As shown in Table 1, the zeta potential of SPIO-Au-RhB NPs was changed from −23.6 mV to −29.8 mV by the functionalization with PCC-2. The increased negative net charge of the particle indicated the successful attachment of PCC-2 with negative net charge. In contrast, the zeta potential of SPIO-Au-Py NPs was changed from −25.1 mV to −14.4 mV by the functionalization with PCC-3. The neutralized net charge of the particle after modification strongly suggested that PCC-3 with positive net charge were on board. When comparing their hydrodynamic diameter before and after modifications, the significantly increased amount again confirmed the successful functionalization with PCC-2 and PCC-3. The large shifts in the lag time as shown in FIG. 4 also indicated the increase of hydrodynamic dimensions from SPIOAu NPs to SPIO-Au-RhB(Py) and to SPIO-Au-RhB(Py)-PCC-2(3). By running the element analysis using ICP-MS, the existence of Au and Fe was further verified by finding a 32.3% total mass of Fe and 67.7% total mass of Au in the stand-alone SPIO-Au NPs, as shown in Table 2. The original data can be found in Table 3. Then a 0.3% total mass of Pd and 3.3% total mass of Co were found respectively in SPIO-Au-Py-PCC-3 and SPIO-Au-RhB-PCC-2, which further demonstrated the successful functionalization of PCC-2 and PCC-3 at the surface of thiol modified SPIO-Au NPs.

TABLE 2
ICP—MS results showing the relative element composition for
the stand-alone SPIO—Au NPs and SPIO—Au NPs functionalized
with PCC-2 and PCC-3. Original data set is listed in Table 3.

| | Fe | Au | Co | Pd |
|---|---|---|---|---|
| | (% Total | (% Total | (% Total | (% Total |
| | Mass) | Mass) | Mass) | Mass) |
| SPIO—Au | 32.3 | 67.7 | N/A | N/A |
| SPIO—Au—Py—PCC—3 | 24.7 | 75.0 | N/A | 0.3 |
| SPIO—Au—RhB—PCC—2 | 22.0 | 74.7 | 3.3 | N/A |

TABLE 3
Mass concentration of Fe, Co, Pd and Au in SPIO-Au NPs
before and after functionalization with PCC-2
and PCC-3 obtained from ICP-MS measurement.

| | Fe (ppb) | Co (ppb) | Pd (ppb) | Au (ppb) |
|---|---|---|---|---|
| SPIO-Au | 3853.7590 | N/A | N/A | 8082.0900 |
| SPIO-Au-Py-PCC-3 | 1596.9500 | N/A | 18.5937 | 4843.9160 |
| SPIO-Au-RhB- | 633.3282 | 94.6915 | N/A | 2144.8960 |
| PCC-2 | | | | |

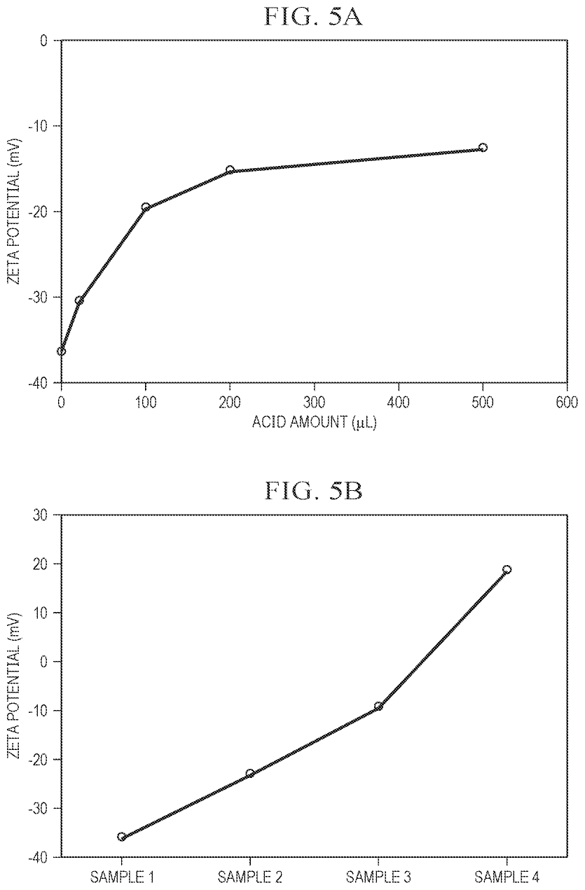

To further adjust the surface charge of PCC-3 functionalized SPIO-Au NPs from negative to positive for the purpose of loading negatively charged RA, the acetic acid was used to neutralize the surface charge of the stand-alone SPIO-Au NPs. As shown in FIG. 5A, by adjusting the acid amount from 20 μL to 500 μL, the zeta potential of SPIO-Au NPs was changed from −36.3 mV to −12.8 mV, showing the neutralization of the negative charged citrate ligands at the surface of SPIO-Au NPs. According to this, it is hypothesized that the overall zeta potential of SPIO-Au-Py-PCC-3 NPs can be adjusted by choosing the appropriate amount of acid to neutralize part of the negatively charged citrate ligand on SPIO-Au NPs before the partial replacement of citrate by Py-PEG-SH at the surface of SPIO-Au NPs. FIG. 5B shows that the surface charge of SPIO-Au NPs was changed from −36.3 mV to −23.3 mV by using the acid neutralization (100 μL of acetic acid), the zeta potential of Py functionalized NPs was then changed to −9.3 mV, and the PCC-3 functionalized NPs was further changed to 18 mV, successfully changing the overall zeta potential of NPs to positive, which makes this nanocarrier capable of attracting RA by the strong electrostatic interaction.

Cellular Uptake Analysis. The cellular uptake of SPIO-Au, SPIO-Au-Py-PCC-3, and SPIO-Au-RhB-PCC-2 NPs was verified at the concentration of 20 μg/mL by TEM analysis on PC-12 cells incubated with NPs for 48 hours. For all three cases, NPs with different functionalization types were found internalized into the PC-12 cells. NPs were found located in the cytoplasm, rather than the nucleus. No NPs were found located in the endosome indicating the different cellular uptake mechanisms from endocytosis. It was possible that the NPs were absorbed into the cells by direct translocation across the cell membrane. More SPIO-Au-Py-PCC-3 NPs were found inside the cells compared with another two cases, implying the promotional effect of PCC-3 functionalization on cellular uptake.

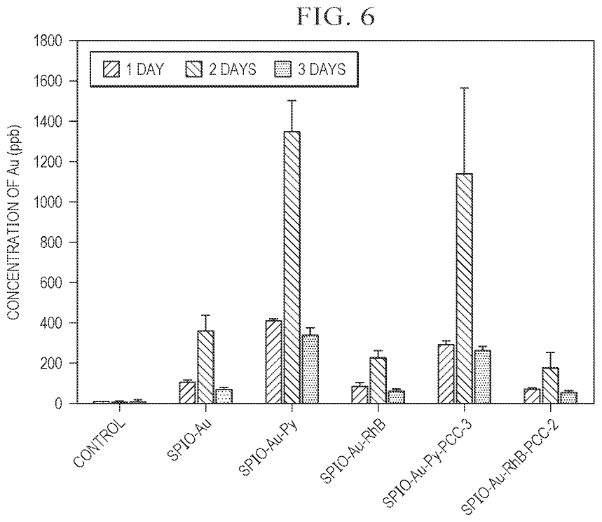

The relative absorbed amount of Au for each cell group treated by NPs with different functionalization types was further measured by running element analysis through ICP-MS at different incubation time points. The Au concentration of each sample group is listed in Table 4. As shown in FIG. 6, at each time point, the Py functionalized and Py-PCC-3 functionalized SPIO-Au NPs gained much higher cellular uptake compared with the stand-alone SPIO-Au NPs, while the functionalization with RhB and RhB-PCC-2 reduced the cellular uptake compared with the stand-alone SPIO-Au NPs. This finding indicated the enhanced cellular uptake by the functionalization of Py and PCC-3, which was in consistent with the TEM results. It was also interesting to notice that the cellular uptake of all kinds of NPs reached the maximum value at 2 days of incubation, suggesting that more NPs were expelled from cells at 3 days of incubation.

TABLE 4
Mass concentration of Au in cells treated with SPIO—Au NPs functionalized
with Py, RhB, PCC-2, and PCC-3 obtained from ICP—MS measurement.

| | | | |
|---|---|---|---|
| | Au Concentration | | |
| | in Independent | Au Concentration in | Au Concentration in |
| | Sample | Independent Sample | Independent Sample |
| group | #1 (ppb) | #2 (ppb) | #3 (ppb) |

1 Day

| | | | |
|---|---|---|---|
| Control | 4.45816283 | 5.46804528 | 6.03148351 |
| SPIO—Au | 114.759258 | 87.7052764 | 116.681768 |
| SPIO—Au—Py | 430.021004 | 400.173842 | 405.881155 |
| SPIO—Au—RhB | 59.109315 | 104.079899 | 97.7110314 |
| SPIO—Au—Py—PCC-3 | 270.44219 | 307.500983 | 304.923798 |
| SPIO—Au—RhB—PCC-2 | 65.7675377 | 76.5870412 | 75.9569287 |

2 Days

| | | | |
|---|---|---|---|
| Control | 0.90774804 | 8.25689555 | 13.9477685 |
| SPIO—Au | 315.892862 | 416.670255 | 408.70618 |
| SPIO—Au—Py | 1515.93561 | 1218.4028 | 1298.56528 |
| SPIO—Au—RhB | 213.87588 | 206.0191 | 269.232583 |
| SPIO—Au—Py—PCC-3 | 842.867031 | 1625.00936 | 953.971484 |
| SPIO—Au—RhB—PCC-2 | 120.039533 | 234.063553 | 1204.99529 |

3 Days

| | | | |
|---|---|---|---|
| Control | 22.8104 | 5.52438334 | 6.08279173 |
| SPIO—Au | 76.05443 | 57.4912211 | 87.7598424 |
| SPIO—Au—Py | 319.9676 | 378.304725 | 329.341497 |
| SPIO—Au—RhB | 64.1323 | 73.5757532 | 71.483045 |
| SPIO—Au—Py—PCC-3 | 234.6405 | 288.471301 | 260.795461 |
| SPIO—Au—RhB—PCC-2 | 46.33348 | 66.043765 | 55.2116213 |

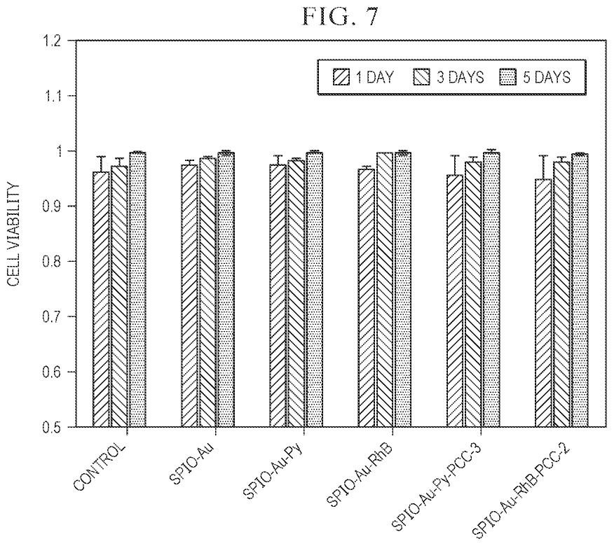

Cellular Viability Examination. To investigate whether or not the functionalization of NPs affects the cellular viability, The PC-12 cells treated with SPIO-Au, SPIO-Au-Py, SPIO-Au-RhB, SPIO-Au-Py-PCC-3, and SPIO-Au-RhB-PCC-2 NPs respectively at the same concentration (20 μg/mL) were incubated for 1, 3, and 5 days. At each time point, the dead cells and total cells were stained by SYTOX™ Green and SYTO™ 59 and analyzed through flow cytometry. As shown in FIG. 7, for up to 5 days of incubation, no significant reduction of cellular viability was observed for all the types of functionalization compared with the control groups with no NPs, indicating the Py, RhB, PCC-2, and PCC-3 functionalization of NPs did not induce any significant toxicity in PC-12 cells. It was also interesting to notice that there was an increase of cell viability for groups treated with NPs at longer incubation time, that is, 5 days. This may be explained by the promotional effect of SPIO-Au NPs on cell proliferation.

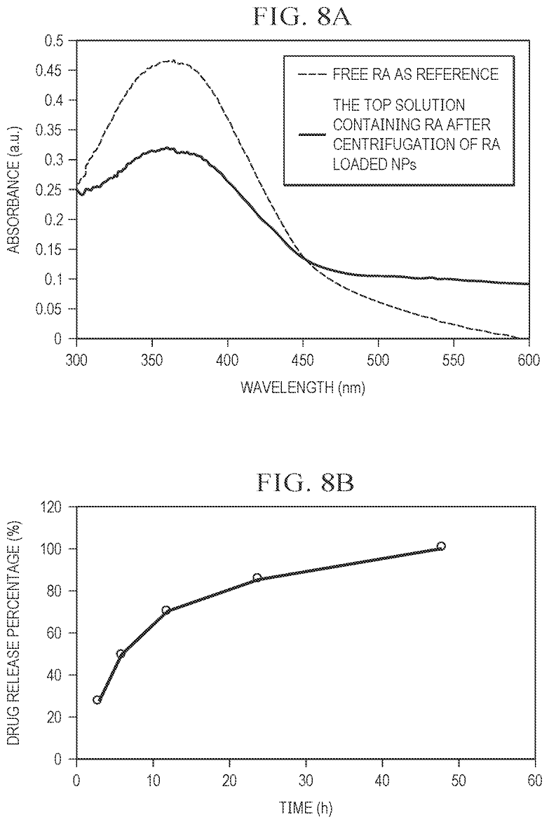

The Drug Loading Capability of PCC-3 Functionalized SPIO-Au NPs. It has been previously found that PCC-2 and PCC-3 could encapsulate organic molecules with positive and negative net charge, respectively. Both electrostatic and hydrophobic interactions contribute to the guest binding. Regarding the structural and functional variety of PCCs, it is useful to select designated ones for specific delivery purpose. Herein, PCC-3 functionalized SPIO-Au NPs with positive net charge was selected for encapsulating the negatively charged moiety of RA. To evaluate the drug loading capability, the surface modified SPIO-Au-Py-PCC-3 NPs with the zeta potential of 18 mV was used as the drug carrier of RA. As shown in FIG. 8A, a significant reduction of the light absorbance was observed in the top solution containing the free unloaded RA compared with the stand-alone RA standard solution, implying that the RA molecules were successfully loaded into the SPIO-Au-Py-PCC-3 NPs. Then the amount the RA loaded to the NPs was further quantified using the RA absorbance value of the top solution and the standard RA solutions according to the Beer-Lambert law. As shown in Table 5, the loading efficiency was estimated to be 31.5%, indicating that about 31.5% of the total fed RA was loaded into the NPs.

TABLE 5
The drug loading characteristics of SPIO—Au—Py—PCC-3

| | | | | RA |
|---|---|---|---|---|
| | SPIO—Au | | Loading | Payload |
| | (mg) | RA (μg) | Efficiency (%) | (μg/mg) |
| Mass | 1.2747 | 9.4 | 31.5 | 7.374 |
| Composition | | | | |
| of Every 1 mL | | | | |
| of Drug Loaded | | | | |
| NPs | | | | |

The RA payload was estimated to be 7.374 μg/mg, indicating that there was 7.374 μg of RA loaded to every 1 mg of SPIO-Au NPs functionalized with Py and PCC-3. The RA release characteristics from SPIO-Au-Py-PCC-3 NPs were shown in FIG. 8B. The release profile of RA included an initial rapid increase period and a relative slow increase period after that. The RA release from NPs was initiated as early as 3 hours, and reached 100% after 48 hours. These data indicated that a sustained release of RA was achieved by SPIO-Au-Py-PCC-3 NPs.

PCC Functionalized SPIO-Au NPs with Tunable Surface Charge and Capping Agents. In the present disclosure, a generalized nanocarrier integrating SPIO-Au core-shell NPs with PCCs using thiol containing molecules (Py(RhB)-PEG-SH) as a bridge has been developed. The configuration of this nanocarrier is based on the hydrophobic internal cavities and net charges of PCC-2 and PCC-3. In aqueous solution, the hydrophobic dye Py can be absorbed into PCC-3 which have strong hydrophobicity. While the cationic dye RhB can be absorbed into PCC-2 with negative net charge due to the electrostatic interactions. The thiol group on the other end of the polymer chain can be attached to the Au surface due to the strong Au—S linkage. Therefore Py-PEG-SH and RhB-PEG-SH can be used to connect SPIO-Au NPs with PCC-3 and PCC-2, respectively. The UV-Vis light absorption results confirmed the successful functionalization of SPIO-Au NPs with RhB-PEG-SH by showing the peak shift towards the typical RhB absorbance peak. The fluorescence measurement confirmed the successful functionalization of SPIO-Au NPs with Py-PEG-SH by showing the typical Py fluorescence emission. The change of zeta potential and hydrodynamic diameter after the treatment with RhB-PEG-SH and Py-PEG-SH and the further treatment with PCC-2 and PCC-3 respectively, strongly demonstrated the successful functionalization at each step with thiol molecules and PCCs. This also indicated that the surface charge of SPIO-Au NPs can be tuned by using different capping ligands to replace the original citrate acid ligands. Their successful functionalization was also confirmed by finding the critical metal element of PCC-3 and PCC-2 through ICP-MS measurement.

PCC Functionalized SPIO-Au NPs as Suitable Platform for Neuronlike Cell Growth. Synthesized SPIO-Au core-shell NPs at 21 nm diameter have been demonstrated to have good in vitro cellular viability on various cell lines including PC-12 cells and MC-3T3 cells at 10 μg/mL to 80 μg/mL dosage level without affecting the cell morphology. However, in some instances, different types of surface functionalization can possibly affect the toxicity of SPIO-Au NPs. Previous studies have found that the surface functionalization or capping ligand may dramatically affect the toxicity of NPs. For example, Au NPs capped by cationic ligands can cause moderate toxicity in vitro. It was also found that citrate- and biotin-functionalized Au NPs were not toxic when used up to at the concentration of 250 mM, whereas cetyltrimethyl ammonium bromide (CTAB)-coated Au NPs were already toxic at 0.05 mM to K562 cells. Therefore, it is useful to evaluate the toxicity of SPIO-Au NPs functionalized with different ligands when they are used as biomaterials or drug carriers. The present disclosure was able to show that the functionalization of Py-PEG-SH and RhB-PEG-SH with NPs did not induce any significant toxicity in PC-12 cells. The further attachment of PCC-2 and PCC-3 to the SPIO-Au NPs also did not affect the viability significantly, indicating the good biocompatibility of this nanocarrier.

It is known that the cellular internalization of NPs can be affected by the factors such as the type of surface charges and the type of functionalization groups of NPs. For example, it has been shown that the cationic Au NPs tend to pass through the cell membrane by direct diffusion, whereas anionic Au NPs enter a cell through endocytosis. For efficient drug delivery, direct diffusion is preferred as NPs tend to aggregate together during endocytosis pathway and are strictly confined to the endosomes. To explore how the different types of surface functionalization of SPIO-Au NPs affect the cellular internalization, the cellular uptake of NPs was visualized through TEM imaging and it was found that the stand-alone SPIO-Au NPs tended to be absorbed by PC-12 cells through direct diffusion. It is noticeable that the PCC-2 and PCC-3 functionalized RhB (Py)-SPIO-Au NPs also tended to be absorbed through direct diffusion, indicating their potential as a nanocarrier for efficient drug delivery. From zeta potential tests, it was found that all these three cases of NPs have negative surface charges, indicating that the similar modifications of NPs using PCCs may have the similar cellular uptake mechanism. However, the different types of thiol groups and PCCs did affect the cellular uptake efficiency. It has been shown that the Py functionalized SPIO-Au NPs were absorbed by PC-12 cells at a much higher amount compared with all other cases including the stand-alone SPIO-Au NPs. With the further functionalization of PCC-3, the absorbed amount was slightly reduced but still much higher than that of the RhB functionalized cases (RhB and RhB-PCC-2) and the stand-alone SPIO-Au case. It is known that the uptake of NPs was mediated mainly by the nonspecific absorption of various serum proteins. It is hypothesized that for Py functionalized NPs there were more types of serum proteins absorbed on their surfaces, which allowed the higher absorption amount into cells via multiple protein receptors.

Effective RA Loading and Release of PCC-3 Functionalized SPIO-Au NPs with Modified Surface Charge. To further demonstrate the ability of this SPIO-Au and PCCs composed nanocarrier for generalized drug delivery purpose by modifying the surface charge, RA was chosen as the guest molecule. The administration of RA in solution form is usually challengeable due to its poor water solubility because of its hydrophobic nature. The half-life of RA is also very short in serum and blood. Using liposomes and deblock copolymers as a drug carrier to improve the water solubility and the half-life of RA has been reported. However, none of the current strategies are designed to fit multiple types of drugs, not to mention the complicated capping process. Thanks to the different hydrophobic inner cavity and the adjustable net charge by choosing the appropriate surface functional groups, the PCCs functionalized SPIO-Au NPs possess the high potential to accommodate the needs of many kinds of drugs. For example, the PCC-2 functionalized SPIO-Au NPs can be used to attract and encapsulate positive charged molecules because of the negative net charge of PCC-2. While the PCC-3 functionalized SPIO-Au NPs can be used to attract and encapsulate the negative charged hydrophobic drugs due to the positive net charge and the stronger hydrophobic internal cavities of PCC-3 compared with PCC-2. Therefore, the present disclosure illustrates the use PCC-3 functionalized SPIO-Au NPs as the drug carrier of RA because of the hydrophobic nature of RA and its negatively charged moiety. For this purpose, the SPIO-Au NPs were firstly surface modified by using acetic acid to neutralize the negative surface charge, which finally led to the shift of the surface charge of PCC-3 functionalized SPIO-Au NPs from negative to positive. This change in surface charge is beneficial of absorbing negatively charged RA. The high loading efficiency and RA payload indicated the strong potential of this PCCs functionalized SPIO-Au NPs not only as the drug carrier of RA, but also the drug carrier of many other drugs, as this customized drug loading strategy can be controlled according to the type of charge and the hydrophobicity of guest molecules. The sustained release pattern of RA from SPIO-Au-Py-PCC-3 NPs suggested the potential of these NPs as a drug carrier of RA to minimize the rapid metabolization of RA in cells. Moreover, the pH-sensitive nature of PCCs also support the controllable drug release of this nanoplatform.

Disclosed above is a novel smart nanocarrier that was developed by preparing PCCs functionalized SPIO-Au NPs using thiol molecules as a bridge. The successful functionalization of SPIO-Au NPs with thiol groups and PCCs was demonstrated using the UV-Vis light absorption measurement, the fluorescence spectroscopy, the zeta potential and the hydrodynamic diameter measurement, as well as the ICP-MS measurement. The cytotoxicity evaluation confirmed the excellent biocompatibility of SPIO-Au NPs (20 μg/mL) functionalized with Py-PEG-SH, RhB-PEG-SH, PCC-3, and PCC-2. The cellular uptake evaluation revealed that the intracellular targeting of PCC-2 and PCC-3 functionalized NPs was achieved mainly by the direct translocation across the cell membranes. The elemental analysis of the cell samples showed the significantly higher cellular uptake of NPs functionalized with Py-PEG-SH. The capability of PCCs functionalized SPIO-Au NPs as a generalized drug-loading-and-release nanoplatform by controlling their surface charges using acetic acid and functional groups with different surface charge and hydrophobicity, which is simple and straightforward, has been demonstrated herein that. Particularly, the effective modification of PCC-3 functionalized SPIO-Au NPs was shown to load RA and the continuous release of RA was observed. Considering the composition of the nanocarrier, it is not only a bio-compatible cargo delivery system, but also a promising intracellular targeting tool and a potential remotely controllable mobile medical device for in vitro and in vivo applications. Furthermore, other designated properties can be incorporated into the nanoplatform by customizing the core-shell part and surface functional modulators. In particular, many multimode medical applications, such as utilizing the magneto-plasmonic nature of SPIO-Au NPs to realize the light/magnetic field-based therapy, are further envisioned.

Furthermore, the following illustrates multifunctional SPIO-Au NPs based smart drug carriers with PCC cages featuring high stability and great drug loading capabilities. SPIO-Au NPs were synthesized and then efficiently functionalized by SH-PEG-Py groups through assistance from the anion exchange resins. This effective surface-exchange approach enables high loading number of cationic PCC cages, which provides great drug-loading capacities. High payload (32.29 μg/mg) was observed, and on-demand release was realized within time frame of NPs' half-life (88% within 24 h). Cell viability experiments confirmed the negligible toxicity of toxicity. The excellent colloidal stability, high drug loading capacity, and fast controlled release profile can provide a solution for neuro-regenerative drug delivery.

Neurodegenerative diseases (NDs), particularly Parkinson's disease and Alzheimer's disease, are projected to affect nearly 7.4 million people in the United States by 2030. Therapeutic agents are developed for the treatment of these diseases, but their efficacy has been largely limited due to ineffective targeted delivery to the brain, such as RA. RA is a molecule utilized in the maintenance of gene expression and acts as a promoter of neuronal cell survival, growth, and differentiation. Because of these intrinsic qualities, RA has been identified as a potential therapeutic agent in the treatment of NDs. Unfortunately, in a clinical context, RA's efficacy has been limited. Reasons include its poor aqueous solubility, its short half-life, and possible side effects when high concentration of it is off-targeted. Another limitation of the intravenous delivery of “free” RA is the low RA accumulation in the brain due to the existence of the BBB, which necessarily restricts the transport of foreign materials into brain tissue. Thus, there is a motivation to develop a strategy for RA delivery and controlled release that would be concentrated at the desired target site. One promising solution is the use of smart nanocarriers, particularly NPs, for the delivery of RA.

Using external stimulation like light, magnetic fields, or focused ultrasound (FUS), NPs can improve the transport of RA across the BBB and extend the blood circulation time of RA. External stimulation in combination with NPs also showed potential for controlled release of drugs. The use of Au NPs has been shown to improve neuronal differentiation, maturation, and growth in PC-12 cells. In addition, SPIO-Au NPs have high biostability, high biocompatibility, inherent magnetic properties, and modifiable surface architectures. These qualities have made SPIO-Au NPs a high-interest candidate for drug delivery systems. When used in conjunction with dynamic magnetic fields (MFs), these SPIO-Au NPs more readily stimulate neuronal growth. However, despite these benefits, most of the existing solutions using NPs have been limited in their ability to target specific cells, difficulties in controlled drug release, and prove impractical in upscale production. These properties compose the major barrier in clinical translation of NP-based treatments.

SPIO-Au NPs conjugated with PCCs capable of reaching 100% release of RA after 96 hours when excited with light emitting diodes (LEDs) have been developed. PCCs have chemical and electrical properties that can be tuned by modifying coordination sites and surface architecture. This benefit allows for the development of PCCs with net-negative, net-positive charges, and hydrophobic inner cavity to be able to flexibly encapsulate hydrophobic molecules. The conjugation of these PCCs with Au-SPIO NPs enabled the RA encapsulation and controlled and sustained release of RA from NPs using light. Furthermore, it was shown that positively charged SPIO-Au-Py-PCC NPs enhance intracellular targeting efficiency. However, these SPIO-Au-PCC NPs suffer from low particle stability, inefficient NP functionalization, and relatively low loading rates.

It has shown that cationic Au NPs accumulate better in the liver and spleen when compared to neutral or anionic NPs, indicating higher biodistribution for cationic NPs. The development of a single-step method for the cationization of Au NPs, which, using anionic exchange resin (AER), proved more stability than their positive or neutral counterparts. AER works by facilitating a ligand exchange reaction of anionic or neutral Au NPs with cationic ligands. A Cl⁻ type AER works by releasing Cl⁻ ions in exchange for citrate from an anionic Au NP, allowing for a cationic thiol to take the citrate's place on the NPs surface. This is a promising solution in improving the stability of Au NPs in general. This simplified procedure for NP cationization also stands to improve the ease of production of Au NPs and prevent aggregation. However, there is still no attempt to use this method to improve drug delivery efficacy with cationic Au NPs.

Inspired by AER assisted method's high efficiency of functionalizing NPs with cations, it was hypothesized that with the assistance of AER methods there would be higher portions of Au-SPIO-PCC surfaces being functionalized with Py-PEG-SH, thus resulting in higher particle stability, and more loading sites for drug molecules. Demonstrated herein, the functionalization process of SPIO-Au NPs with SH-PEG-Py ligands using AER as catalysts for surface-exchange reaction has been synthesized and improved. Being decorated with more ligands, more cationic PCC-3 cages were able to be anchored onto SPIO-Au NPs, which successfully improved the particle stability with higher surface charge, and increased loading rate of RA. To demonstrate the on-demand release of RA under light stimulation, in vitro drug release experiments were carried out using a 100-mW laser (525 nm) as an irradiation source. The release rate of RA was tested to be ˜88% after only 24 hours with the periodically applied laser. Cytotoxicity study has demonstrated that SPIO-Au-PCC NPs have minimal toxicity when introduced to PC-12 cells.

Materials. The chemicals below were obtained from corresponding manufacturers and used as received. SPIO NPs (EMG 304, Ferrotec, Santa Clara, CA), trisodium citrate (Na₃C₆HSO₇, 99%, Alfa Aesar, Haverhill, MA), chloroauric acid (HAuCL₄, >99.0%, Sigma-Aldrich, St. Louis, MO), AMBERLITE™ IRA-410 chloride form anion exchange resin (Sigma-Aldrich, St. Louis, MO), ammonium acetate (Sigma-Aldrich, St. Louis, MO). SPIO-Au NPs and PCC-3 were prepared.

PC-12 cells (ATCC, Manassas, VA) were cultured in RPMI-1640 Media with ATCC modification (Life Technologies, Carlsbad, CA) containing dilutions of 10% HI horse serum (Gibco, Thermo Fisher Scientific), 5% heat-inactivated fetal bovine serum (HI FBS; Gibco, Thermo Fisher Scientific, Waltham, MA), and 1% Penicillin-Streptomycin (Gibco, Thermo Fisher Scientific, Waltham, MA). Materials included collagen from human placenta, Bornstein and Traub Type IV, BioReagent (Sigma-Aldrich, St. Louis, MO), staining with SYTO™ 59 (Invitrogen, Thermo Fisher Scientific, Waltham, MA), staining with SYTOX™ Green (Invitrogen, Thermo Fisher Scientific, Waltham, MA), washing with 1×HYCLONE™ PBS (Cytiva, Marlborough, MA), and disassociation with TRYPLE™ (Gibco, Thermo Fisher Scientific, Waltham, MA).

Functionalization of SPIO-Au NPs with Py-PEG-SH and PCC-3 via Direct Exchange Method. SPIO-Au NPs were functionalized firstly with Py-PEG-SH by suspending 1000 μg of SPIO-Au NPs in 1 mL of DI water with 1500 μg of Py-PEG-SH and shaking on shaker at room temperature for 24 hours. Then the Py-PEG-SH functionalized SPIO-Au NPs (named as SPIO-Au-Py) were centrifuged and washed by DI water for three times. After that, SPIO-Au-Py NPs were suspended in 1 mL DI water and mixed with 450 μg of PCC-3 at room temperature for 24 hours, followed by centrifuging and washing process for three times with DI water.

Functionalization of SPIO-Au NPs with Py-PEG-SH and PCC-3 via AER Assisted Method. SPIO-Au NPs were functionalized firstly with Py-PEG-SH by suspending 1000 μg of SPIO-Au NPs and 1.667 g of AER in 1 mL of DI water with 1500 ug of Py-PEG-SH and shaking on shaker at room temperature for 2 hours. The solution was taken out first to remove the AER. Then the Py-PEG-SH functionalized SPIO-Au NPs (named as SPIO-Au-Py) were centrifuged and washed by DI water for three times. After that, SPIO-Au-Py NPs were suspended in 1 mL DI water and mixed with 450 μg of PCC-3 at room temperature for 24 hours, followed by centrifuging and washing process for three times with DI water.

Characterizations. TEM analysis was performed on a Tecnai G2 F20 ST FE-TEM (FEI) at an operating voltage of 200 kV. Briefly, 20 μL of each sample was dropped onto a 400-mesh copper grid (Electron Microscopy Sciences, Hatfield, PA) and then left dry in the air. The zeta potential and the hydrodynamic diameter of SPIO-Au NPs with different functionalization was measured with a Zetasizer Nano ZS (Malvern Instruments Inc.).

Elemental Analysis. The SPIO-Au NPs before and after functionalization with PCCs were digested in aqua regia. In detail, the NPs were digested by a mixture of concentrated hydrochloric acid (0.3 mL, ˜30%) and concentrated nitric acid (1.1 mL, ˜65%). The samples were then diluted with 2% nitric acid and finally tested on the NEXION@ 300D ICP-MS (PerkinElmer, Inc.).

Photothermal Conversion Evaluation. To evaluate the photothermal conversion effect, the same setup for the in vitro drug release was used to reflect the real temperature change under in vitro experimental conditions. A GX3 high power green laser pointer was used as light source (operation power: 100 mW). SLIDE-A-LYZERrm MINI dialysis device kit (10K molecular weight cutoff (MWCO), 0.1 mL) was used as the container, as 1.2 mL of DI water and a mini stir bar was placed in the micro centrifuge tube and certain volume of SPIO-Au-Py-PCC NPs with certain concentration was placed in the dialysis tube. A thermocouple (Omega, Stamford, CT, K-type, 0.076 mm wire diameter, and 0.33 mm bead diameter) was used to measure the temperature variation with time upon laser irradiation. A National Instruments NI9219 control system was used to record the temperature variation curve with time. The stirring was set to 300 rpm. The laser irradiation was started when the temperature established equilibrium at room temperature. The laser was turned off when the raised temperature stabilized.

Measurement of the Drug Loading Efficiency. 0.25 mg SPIO-Au-Py-PCC NPs were mixed with 4 μg RA in 1.25 mL DI water-methanol mixed solvent (1:1, v:v). The tube was shaken under dark for 24 hours. After the NPs were centrifuged and separated from the solvent, RA-loaded SPIO-Au-Py-PCC NPs were obtained. The loaded amount was calculated by RA's concentrations (tested by high-performance liquid chromatography; HPLC) before and after the loading process. The payload was calculated via Equation 1, shown above.

Determination of the In Vitro Drug Release Profile. The drug release profile of RA loaded SPIO-Au-Py-PCC NPs was measured in DI water. The NPs were dispersed in 200 μL of DI water and then introduced into the micro dialysis tube (10K MWCO). A dialysis tube was then placed into a microcentrifuge tube containing 1.2 mL of DI water and a mini stir-bar. The stirring was set to 300 rpm during the release process. Laser light of 100 mW intensity was applied to the dialysis tube for 10 minutes between two consecutive time points. For time intervals longer than 6 hours, the laser was applied in total for three times. At each time point, all the released media were exchanged with fresh DI water. The media with RA released within each time interval was measured by HPLC. The control group did the sampling in the same way, as no external light was applied.

Analysis of RA using HPLC. RA was measured by HPLC (HPLC-2030C, Shimadzu) equipped with a reversed-phase C18 column in the low-pressure gradient mode. A mixture of methanol and 10 mM ammonium acetate (75:25, v:v) was used as the mobile phase at a flow rate of 1.0 mLmin. The column effluent was monitored using an ultraviolet (UV) detector set at 350 nm. RA stock solutions were prepared by RA and methanol, and then stocked in a freezer under dark. Calibration curves were obtained from RA-methanol solutions over the range 0.3 to 190 μg/mL. The curve was linear and R² was 0.996.

Cell Viability. The cell viability evaluation used a flow cytometry method. PC-12 cells were seeded into a 48-well plate pre-coated with collagen type IV. The cells were then incubated at 5% CO₂ and 37° C. After 24 hours, the cells were incubated with media containing SPIO-Au-Py-PCC NPs at concentrations between 0 μg/mL and 40 μg/mL. After 1, 3, and 5 days, the media was aspirated and the cells were stained with 200 μL of 5 μM SYTOX™ Green and SYTO™ 59 (1:1000 dilution from stock solution, Invitrogen) for 10 minutes. Cells were then observed through and imaged using an EVOS™ microscope at 10× magnification. The staining solution was removed, and the cells were washed with PBS, treated with TRYPLE™ for 10 minutes, and then resuspended in media for fluorescence measurement using a BD ACCURI™ C6 flow cytometer equipped with a FL1 (553 nm) and FL4 detector. Viability data was acquired at a flow rate of 35 μL/min with a minimum of 10,000 events detected.

Synthesis and Functionalization of SPIO-Au NPs. The synthesis of SPIO NPs and SPIO-Au NPs was carried out. The functionalization of SPIO-Au nanoparticles was performed using an anion-exchange resin assisted method, with SH-PEG-Py ligands being used as surface decorators. The anion exchange resin beads were firstly mixed with the diluted SPIO-Au NPs suspension. SH-PEG-Py solution were then added in the suspension under ultra-sonication for a better mixing. After AER beads are simply filtered out, the resulted SPIO-Au-Py NPs were then separated with centrifuge. SPIO-Au-Py NPs maintain the same color as ones before the functionalization, while the stability has been improved. No precipitates were observed even after NPs being set rest for a week. After that, PCC-3 cages were anchored with pyrene groups on SPIO-Au-Py NPs through simple mixing, resulting in SPIO-Au-Py-PCC NPs, with the potential of carrying drugs.

Characterization of Functionalized SPIO-Au NPs. TEM images of SPIO-Au-Py-PCC NPs showed the SPIO-Au-Py-PCC NPs with a quasi-spherical shape and a narrow size distribution. The mapping results showed the presence of Au and Fe, which confirmed the core-shell structure. No aggregation was observed from TEM images.

To quantify the effect that the AER assisted method brought, the zeta potential and hydrodynamic diameters of NPs were tested. Effects of different usage of surface decorators were studied. Defining the NPs functionalized by calculated stoichiometric dose of SH-PEG-Py defined as SPIO-Au-Py-1, NPs decorated with 50% more and double doses were also prepared. As shown in Table 6A, a significant change of surface charge was observed due to the functionalization by SH-PEG-Py. It jumped from −44.20 mV to 34.77 mV (SPIO-Au-Py-1) and then 36.00 mV (SPIO-Au-Py-1.5), indicating the high-efficiency displacement of surface citrates by SH-PEG-Py ligands. Comparing to the hydrodynamic size (60.19 nm) of citrated SPIO-Au NPs, diameters of SPIO-Au-Py NPs expanded due to the expanding volume from the SH-PEG-Py ligands. Excessive amount of SH-PEG-Py is not favored as it caused decrease in zeta potential and increase in hydrodynamic size.

Different dosages of PCC cages also alternate the outcomes (Table 6B). The highest surface charge and smallest hydrodynamic diameter were achieved on SPIO-Au-Py-1.5-PCC-2, which were 39.97 mV and 118.60 nm. However, the results from SPIO-Au-Py-1.5-PCC-1.5 were also close. Regarding the conservation of PCC cages, the condition of SPIO-Au-Py-1.5-PCC-1.5 was picked as the standard preparation procedure of drug carriers.

Tables 6A-6B. Z-Potentials and hydrodynamic diameters of SPIO-Au NPs with different functionalization with AER.

TABLE 6A
Different Dosage of SH—PEG—Py Ligands

| NPs | SPIO—Au | SPIO—Au—Py-1 | SPIO—Au—Py-1.5 | SPIO—Au—Py-2 |
|---|---|---|---|---|
| Zeta Potential | −44.20 ± 0.18 | 34.77 ± 0.82 | 36.00 ± 1.28 | 30.27 ± 0.62 |
| (mV) | | | | |
| Hydrodynamic | 60.19 ± 1.02 | 148.40 ± 1.47 | 120.67 ± 1.68 | 257.00 ± 5.39 |
| Diameter (nm) | | | | |

TABLE 6B
Different Dosage of PCC-3 Cages

| | SPIO—Au—Py- | SPIO—Au—Py- | SPIO—Au—Py- | SPIO—Au—Py- |
|---|---|---|---|---|
| NPs | 1.5 | 1.5-PCC-1 | 1.5-PCC-1.5 | 1.5-PCC-2 |
| Zeta Potential | 36.00 ± 1.28 | 29.10 ± 1.96 | 38.90 ± 0.71 | 39.97 ± 1.07 |
| (mV) | | | | |
| Hydrodynamic | 120.67 ± 1.68 | 148.60 ± 1.37 | 123.13 ± 1.60 | 118.60 ± 0.73 |
| Diameter (nm) | | | | |

To better understand the components of functionalized SPIO-Au NPs, element analysis was performed (Table 7). The concentration of Au, Fe, and Pd reflects the concentration of Au shell, SPIO core (Fe₃O₄), and PCC-3 cages (Pd exists in their building units), respectively. The mass ratio between Au and Fe remained the same (Au:Fe, 4:1, m:m). The high mass ratio of Pd (10.2%) proved the successful and efficient combination of PCC-3 cages onto the NPs. Comparing to the elemental analysis of NPs functionalized by direct replacement method, the improvement in the mass rate PCC-3 was significant.

TABLE 7
Relative mass components of SPIO—Au and SPIO—Au—Py—PCC
NPs. Results were obtained from ICP-MS analysis.

| NPs | Fe/ % | Au/ % | Pd/ % |
|---|---|---|---|
| SPIO—Au | 19.2 | 80.8 | — |
| SPIO—Au—Py—PCC | 17.2 | 72.6 | 10.2 |

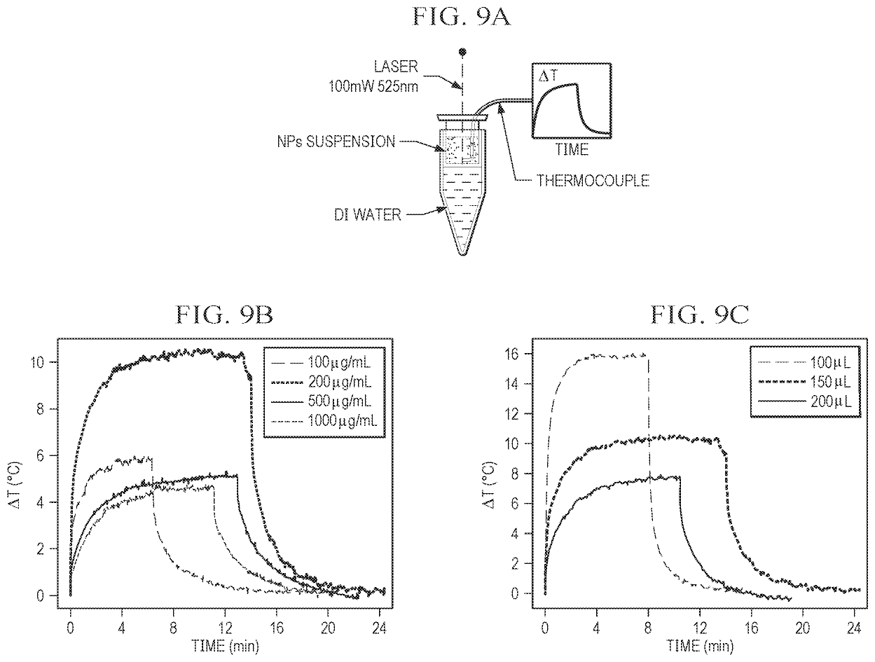

Photothermal Conversion. The photothermal conversion effect plays a role in controlled release of drugs from carriers. To examine the photothermal response of SPIO-Au-Py-PCC NPs for a better understanding of their behavior in situ, the measurement was set up in the apparatus for in vitro release study (FIG. 9A). The incident wavelength of laser was set to 525 nm to match the surface plasmonic resonance peak of SPIO-Au. The power of incident laser was 100 mW.

The temperature profile of SPIO-Au NPs with different concentrations (FIG. 9B) were measured first. Even though a higher concentration of NPs can carry more drug, it will drastically reduce the depth the laser can penetrate and will cause the overall heating effect much weaker. The volumes of different NPs solutions were all set to 150 μL for direct comparison. The laser caused a temperature rise of around 10.6° C. after 10.5 minutes when concentration is 200 μg/mL. NPs of higher concentrations (500 μg/mL, 1000 μg/mL) achieved lower temperature rise (4.7° C., 5.3° C., respectively), which indicates that the laser cannot reach all NPs efficiently.

With the optimal concentration (200 μg/mL) used, the temperature profile of SPIO-Au NPs with different volumes (FIG. 9C) were then measured. The measured thermal variation curves showed positive correlation between volume and maximum temperature rise when equilibrium established. It can be attributed to the increasing path length for laser to reach the bottom as the volume increases. However, temperature increases of 16° C. was too high for the drug delivery and on-demand treatment purpose, 200 μL was selected as the optimal volume for in vitro study due to the moderate temperature rise (7.9° C.).

Drug Loading and In Vitro Release Study. The drug loading capacity of SPIO-Au-Py-PCC are proposed to be improved with the assistance of AER, as more PCC-3 cages were anchored on the nanoparticles when more surface area were functionalized with the SH-PEG-Py ligand. The host-guest interaction between hydrophobic PCC-3 cages and hydrophobic retinoic acid made the loading of RA into PCC-3 favored. Additionally, the high positive charge of NPs also promotes the loading, as retinoic acids can partially ionize into anions and thus be attracted by the NPs.

The loading rate of RA was determined by measuring the concentration difference of RA solution before and after the loading by HPLC. The RA's loading rate was determined to be 32.29 μg/mg NPs. This directly demonstrated that the AER assisted method can lead to a much higher RA loading capacity comparing with the direct replacement method (13.51 μg/mg NPs).

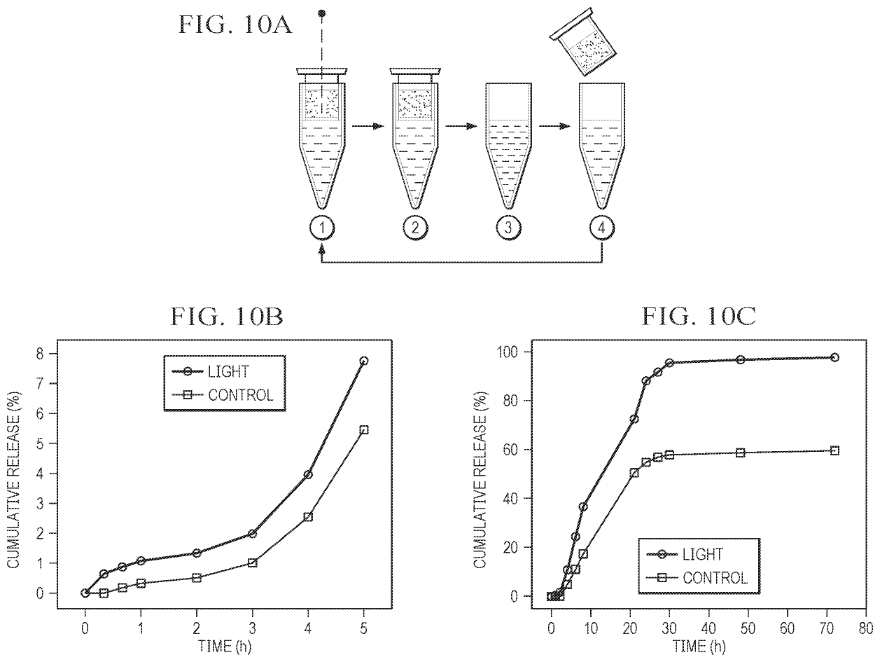

The in vitro release of RA from RA-loaded SPIO-Au-Py-PCC NPs were then measured. A dialysis tube was used to help the separation of NPs from the solution when taking samples. 200 μL of 200 μg/mL SPIO-Au-Py-PCC NPs (calculated based on SPIO-Au) was loaded into dialysis tubing, with 1.2 mL of DI water loaded as solvent in the tube. After each time period, the solvent with the released was collected for analysis, the tube was refilled with fresh DI water to start the next illumination by laser (FIG. 10A). The light group was irritated by 100 mW 525 nm laser for 10 min at each time interval (three times for longer time periods), while the controlled group remained untreated.

The cumulative RA release profile from SPIO-Au-Py-PCC NPs showed significant difference between the light and control group (FIG. 10B). Around 87.9% of loaded RA was released after a total 24 hours' period, which was much higher than control group's 54.8% release efficiency. Being extended to 72 hours, around 97.6% of RA was released in the light group, still more than 40% of RA remained unreleased without laser treatment.

The initial stage of RA release profile from SPIO-Au-Py-PCC NPs was also measured to help understand the kinetical behaviors (FIG. 10C). The initial release rate remained low and steady in the first three hours, with an acceleration occurred starting after that. Such delayed release could be attributed to the size effect of RA and strong interactions between the PCC-3 cages and RA molecules.

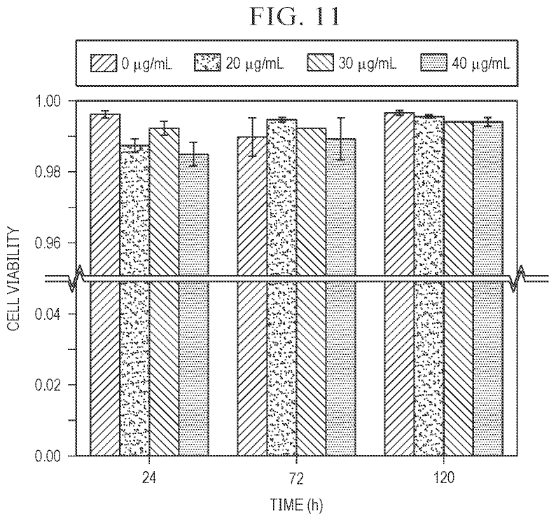

Cellular Toxicity Study. To determine the impact of SPIO-Au-Py-PCC on in vitro cell viability, PC-12 cells were treated with NPs at concentrations between 0 μg/mL and 40 μg/mL. Over the 5 days of incubation, cell viability was largely unaffected. There was no significant impact on the live-to-total cell ratio for all NP-treated groups when compared to the control group. This indicates that SPIO-Au-Py-PCC NPs, at the aforementioned concentrations, do not induce significant toxic effects on PC-12 cells (FIG. 11).

As demonstrated herein, the target is to build light-responsive drug carriers that are with good stability and drug loading capacity and has the potential to realize the on-demand delivery in desired period. The SPIO-Au NPs were synthesized and were then efficiently functionalized with SH-PEG-Py ligands and PCC-3 cages using an anion exchange resin assisted method. The good bioavailability was confirmed by both characterizations (TEM, zeta potential, and hydrodynamic diameters) and in vitro cell toxicity study. Loading rate of RA was tested to be high, and complete on-demand release was achieved through periodically applying of the laser light source.

One of the problems solved was the improvement of the functionalization efficiency of SPIO-Au NPs. The SPIO-Au NPs were synthesized with protection provided by anionic citrates to achieve the uniform size distribution. Since the desired product will be functionalized by cationic PCC-3, one difficulty is to turn anionic SPIO-Au NPs to cationic smoothly. The direct exchange of surface ligands, which can be realized by simply mixing the citrate stabilized NPs with cationic ligands, can raise the surface charge to neutral and then lead to severe and irreversible aggregation, which should be avoided. Even in previous works, this problem was seen in the functionalization of SPIO-Au NPs. The directly synthesized SPIO-Au-Py-PCC NPs cannot pass the threshold of 0 mV even with excessive SH-Py-PEG ligands and PCC-3 cages being used. The calculated number of PCC-3 cages decorated on each NP was low and thus limited the loading capacity. Furthermore, the near-neutral surface charge harms the stability and bioavailability as well. Recently, a rapid and efficient surface exchange of citrate-protect Au NPs with cationic thiol ligands under the presence of AER in water was reported. The AER absorbed the citrates that has been replaced from the surface of Au, pushed the exchange reaction forward to a complete end as the removal of products (citrates) dropping from the reactants makes the replacement direction thermodynamically favored. The kinetics of this method was also reported to be fast as only 20 minutes could lead the surface exchange to completion.

With the help of AER, as shown herein, the efficiency of surface functionalization of SPIO-Au NPs was greatly improved. The highest surface charge achieved was 39.97±1.07 mV for SPIO-Au-Py-1.5-PCC-2, which was far higher than the surface potential of NPs functionalized through direct exchange method (−16.9±4.6 mV). The relatively small hydrodynamic size of as-functionalized NPs (118.60±0.73 nm) also demonstrated the good aqueous stability and potentially excellent bioavailability. The elemental analysis results (10.2% of Pd's mass rate) determined the significantly higher (comparing to previous 0.3% mass rate of Pd) numbers of PCC-3 combined with NPs, indicating the success of complete surface decoration.

A higher loading rate of RA on NPs assisted in reducing the potential toxicity to cells caused by high dosage of NPs and the cost of materials. This was benefitted from the higher loading number of PCC-3 cages, which contributed higher level of interaction between PCC-3 cages' hydrophobic inner cavities and hydrophobic RA molecules. The higher positive charge of NPs also promoted the coulombic attraction between cationic NPs and anions dissociated from RA molecules. Compared to the previously reported payload (13.51 μg/mg), an 139% improvement was observed, which proved the efficacy of this methodology.

To summarize, disclosed herein is an effective approach to build drug loading units-enriched smart nanocarriers from PCCs and SPIO-Au NPs with assistance of AER. The 32.39 μg/mL high payload and ˜88% drug release observed after 1-day treatment has demonstrated potential for on-demand drug delivery purposes. The as-synthesized drug carriers have the features of high positive charge, great colloidal stability, good photothermal response, and minimal toxicity, which solves the problems of neuro-regenerative delivery.

In view of the above, in various embodiments, the present disclosure relates to a method of delivering a drug. Such methods generally include one or more of the following steps of: (1) administering a nanocarrier according to aspects of the present disclosure to a subject; (2) targeting, by the nanocarier, an area in the subject; and (3) releasing a composition having the drug to the area. In some embodiments, the releasing includes exciting the nanocarrier with a light source to release the drug. In some embodiments, the drug is loaded into a PCC attached to the shell via a bridge.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A nanocarrier comprising:
a shell; and
a core disposed within the shell, wherein the shell comprises a functionalized surface, wherein the nanocarrier is selected from the group consisting of SPIO-Au, SPIO-Au-RhB, SPIO-Au-RhB-PCC-2, SPIO-Au-Py, SPIO-Au-Py-PCC-3, and combinations thereof.

2. The nanocarrier of claim 1, wherein the functionalized surface comprises at least one porous coordination cage (PCC) attached to the shell via a bridge.

3. The nanocarrier of claim 2, wherein the PCC comprises a metal cluster and an organic linker.

4. The nanocarrier of claim 2, wherein the bridge is a compound comprising a thiol.

5. The nanocarrier of claim 2, wherein the bridge is pyrene-(rhodamine B)-(polyethylene glycol)-SH (Py(RhB)-PEG-SH).

6. The nanocarrier of claim 2, wherein the PCC is at least one of PCC-2 or PCC-3

7. The nanocarrier of claim 1, wherein the shell is a metal shell.

8. The nanocarrier of claim 1, wherein the core is a superparamagnetic iron oxide (SPIO).

9. The nanocarrier of claim 1, wherein the functionalized surface encapsulates a compound.

10. The nanocarrier of claim 1, wherein the nanocarrier is modified to penetrate a cytoplasmatic membrane by direction translocation.

11. The nanocarrier of claim 1, wherein at least of an anionic exchange resin assisted method or a direct exchange method forms the functionalized surface of the shell.

12. The nanocarrier of claim 1, wherein a drug loaded into a PCC attached to the shell via a bridge is released upon excitation of the nanocarriers via a light source.

13. (canceled)

14. A method of drug delivery, the method comprising:
administering a nanocarrier to a subject;
targeting, by the nanocarrier, an area in the subject; and
releasing a composition comprising the drug to the area, wherein the nanocarrier comprises:
a shell; and
a core disposed within the shell, wherein the shell comprises a functionalized surface.

15. The method of claim 14, wherein the functionalized surface comprises at least one porous coordination cage (PCC) attached to the shell via a bridge, and further wherein the PCC comprises a metal cluster and an organic linker.

16. (canceled)

17. The method of claim 15, wherein the bridge is a compound comprising a thiol.

18. The method of claim 15, wherein the bridge is pyrene-(rhodamine B)-(polyethylene glycol)-SH (Py(RhB)-PEG-SH).

19. The method of claim 15, wherein the PCC is at least one of PCC-2 or PCC-3

20. The method of claim 14, wherein the shell is a metal shell.

21. The method of claim 20, wherein the metal shell is gold (Au).

22. The method of claim 14, wherein the core is a superparamagnetic iron oxide (SPIO).

23-26. (canceled)
